(19) United States
(12) Patent Application Publication
(10) Pub. No.: US 2015/0086602 A1
(43) Pub. Date: Mar. 26, 2015

(54) METHODS FOR COATING BONE ALLOGRAFTS WITH PERIOSTEUM-MIMETIC TISSUE ENGINEERING SCAFFOLDS

(71) Applicant: COLORADO STATE UNIVERSITY RESEARCH FOUNDATION, Fort Collins, CO (US)

(72) Inventors: Matthew J. Kipper, Fort Collins, CO (US); Nicole P. Ehrhart, Fort Collins, CO (US); Raimundo Romero, Fort Collins, CO (US); Timothy R. Gonzales, Wellington, CO (US)

(21) Appl. No.: 14/496,992

(22) Filed: Sep. 25, 2014

Related U.S. Application Data
(60) Provisional application No. 61/882,477, filed on Sep. 25, 2013.

(51) Int. Cl. A61L 27/26; A61L 27/20; A61L 27/34; A61L 27/36; A61L 27/54

(52) U.S. Cl. CPC A61L 27/26; A61L 27/365; A61L 27/54; A61L 27/3608; A61L 27/34; A61L 27/20; A61L 2430/02; A61L 2430/40; A61L 2420/02; A61L 2420/08; A61L 2420/06; A61L 2400/18; A61L 2300/602; A61L 2300/61; A61L 2300/414; A61L 2300/412; A61L 2300/252
USPC 424/423; 514/8.9; 514/9.1; 427/2.26

(57) ABSTRACT

Methods of coating bone surfaces and coated bones comprising a coating comprising (a) a porous polysaccharide scaffold and/or a plurality of polysaccharide nanofibers; and (b) a polyelectrolyte multilayer composition, are disclosed. The methods of coating bone surfaces and the coated bones disclosed provide a biomimetic periosteum to compensate for periosteum lost in preparing bone grafts, and the coated bone grafts, including allografts, restore lost osteogenic and osteoinductive qualities and improve clinical outcomes.

### CROSS-REFERENCE TO RELATED APPLICATION

This application claims the benefit of priority of U.S. Provisional Patent Application No. 61/882,477, filed Sep. 25, 2013. The disclosure of the priority application is incorporated herein by reference.

### FIELD OF DISCLOSURE

The disclosure relates to methods of coating bone surfaces and coated bones for use in bone grafts and for delivery of therapeutic agents including growth factors.

### BACKGROUND

The inability of bone to heal large defects necessitates the use of bone grafts in many traumatic injuries and disease states. Estimates of the number of bone graft procedures performed annually in the U.S. range from 500,000 to 600,000 (in 2002) to 1.5 million (in 2008) (Bucholz R W. Clin Orthop Rel Res. 395:44-52. 2002; Jahangir et al. AAOS Now. January, 2008 ed. Rosemont, Ill.: American Academy of Orthopaedic Surgeons; 2008). Bone autografts, wherein the patient's own bone is used for the transplantation, are considered the gold standard for treatment. The superior clinical performance of autografts can be attributed in part to the preservation of the periosteum, the membrane covering the outer bone surface. The periosteum has been shown to be a critical component of bone healing due to its high vascularization, osteogenic progenitor cells, osteoinductive growth factors, and an osteoconductive structure (Colnot et al. J Orthopaedic Research. 30:1869-78. 2012; Zhang et al. J Bone Miner Res. 20:2124-37. 2005). Autograft bone heals by a process that is very similar to fracture healing and is characterized by initial inflammation and vascularization, followed by recruitment of mesenchymal stem cells (MSCs), which differentiate into osteoblasts and chondroblasts. However, bone autografts are not without limitations. Autografts have limited graft size availability, and donor site morbidity associated with the autograft harvest preclude autograft use in many cases or can lead to further complications such as pain and infection.

Bone allografts, wherein the donor and recipient of the bone graft are different individuals from the same species, are a viable clinical alternative, as they avoid some of the limitations of autografts. Bone allografts are an attractive alternative to autografts and non-biologic endoprostheses because of their potential to integrate with the host and subsequently restore normal limb function without the morbidity associated with the harvest of autografts. In order to mitigate a host-allograft immune response and disease transmission, bone allografts must undergo rigorous cleansing and sterilization steps before implantation, which includes removal of the periosteum. The removal of the periosteum, and its osteoprogenitor cells and osteoinductive factors critical to natural bone healing, leads to suboptimal clinical performance and severely diminishes osteogenic potential (Bauer et al. Clinical Orthopaedics and Related Research. 371:10-27. 2000). This limited healing capacity often results in premature failure of allografts. Resultantly, the failure rate of segmental bone allografts at 10 years has been documented as high as 60% (Yazici et al. Biomaterials. 29:3882-7. 2008). Clearly, novel strategies are needed to improve the osteogenic and osteoinductive characteristics of bone allografts and allograft incorporation.

### SUMMARY

The disclosure relates to bone surfaces having a biomimetic periosteum coating with osteogenic and osteoinductive properties. The coated bones may be used as bone grafts for transplantation and to deliver therapeutic agents including growth factors.

In one aspect, the disclosure provides a coated bone comprising a coating comprising (a) a porous polysaccharide scaffold and/or a plurality of polysaccharide nanofibers; and (b) a polyelectrolyte multilayer composition. In some embodiments, a porous polysaccharide scaffold is freeze-dried onto the bone surface. In other embodiments, a plurality of polysaccharide nanofibers is electrospun onto the bone surface. In some embodiments, the polyelectrolyte multilayer composition is contacting the porous polysaccharide scaffold surface and/or the plurality of polysaccharide nanofibers. In some embodiments, the coated bone is a bone graft, for example, a bone allograft.

In another aspect, the disclosure provides a method of stabilizing and delivering a therapeutic agent, for example, a growth factor, a progenitor cell, a hormone, an anti-inflammatory agent, an antibiotic, a polynucleotide, and/or a chemotherapeutic agent, comprising administering a coated bone described herein to a subject in need thereof. In some embodiments, the therapeutic agent is a growth factor, and the growth factor binds to a polyanion in the polyelectrolyte multilayer composition, which can stabilize the growth factor and potentiate its activity.

In yet another aspect, the disclosure provides a method of coating a bone surface comprising contacting the bone with (a) a porous scaffold-forming polysaccharide and/or a plurality of polysaccharide nanofibers and (b) a polyelectrolyte multilayer composition. In some embodiments, the porous scaffold-forming polysaccharide is deposited onto the bone surface to form a porous polysaccharide scaffold, e.g., by freeze-drying, and the polyelectrolyte multilayer composition is applied onto the porous polysaccharide scaffold. In other embodiments, the plurality of polysaccharide nanofibers is deposited onto the bone surface, e.g., by electrospinning, and the polyelectrolyte multilayer composition is applied onto the plurality of polysaccharide nanofibers.

In another aspect, the disclosure provides a kit comprising a porous scaffold-forming polysaccharide and/or a nanofiber-forming polysaccharide; (b) a polyelectrolyte multilayer-forming composition; and (c) instructions for coating a bone surface with (a) and (b).

In various embodiments of the compositions and methods of the present disclosure, the porous polysaccharide scaffold and/or plurality of polysaccharide nanofibers comprises a polysaccharide selected from the group consisting of chitosan, acylated chitosan, alkylated chitosan, and combinations thereof. In related embodiments, the polyelectrolyte multilayer composition comprises a polyanion selected from the group consisting of a glycosaminoglycan (e.g., heparin, heparan sulfate, hyaluronan, chondroitin sulfate, dermatan sulfate, or keratan sulfate), an anionic polysaccharide, a synthetic anionic polymer, and combinations thereof; and a polycation selected from the group consisting of chitosan, acylated chitosan, alkylated chitosan, poly-lysine, polyethylenimine, and combinations thereof. In some embodiments, the polyelectrolyte multilayer composition comprises N,N,N,-trimethyl chitosan and heparin.

Other features and advantages of the present disclosure will become apparent from the following Detailed Description, including the drawings. It should be understood, however, that the Detailed Description and the specific Examples, while indicating preferred embodiments, are provided for illustration only because various changes and modifications within the spirit and scope of the invention will become apparent to those skilled in the art from the Detailed Description.

### BRIEF DESCRIPTION OF THE DRAWINGS

FIG. 1A shows the chemical structures of chitosan, N,N,N-trimethyl chitosan (TMC), and heparin. FIG. 1B shows a schematic of the different surface coating methods used to form the bone surface coatings of the present disclosure.

FIG. 2 shows a schematic of a collection apparatus for electrospinning a plurality of polysaccharide nanofibers directly onto bone allografts.

FIG. 3A-I shows scanning electron micrographs of (top row) (FIG. 3A) cortical bone, (FIG. 3B) cortical bone coated with phosphonoundecanoic acid (PUA), and (FIG. 3C) cortical bone coated with PUA and a TMC-heparin polyelectrolyte multilayer (PEM); (middle row) (FIG. 3D) cortical bone coated with a chitosan freeze-dried (FD) scaffold, (FIG. 3E) the same FD scaffold after ammonium hydroxide neutralization, and (FIG. 3F) after TMC-heparin PEM deposition; (bottom row) (FIG. 3G) cortical bone coated with electrospun chitosan nanofibers (NF), (FIG. 3H) the NF after ammonium hydroxide neutralization, and (FIG. 31) after TMC-heparin PEM deposition.

FIG. 4 shows high-resolution X-ray photoelectron spectra of the Ca2p, S2p, and P2p envelopes of cortical bone before and after TMC-heparin PEM deposition. Attenuation of calcium and phosphorus signals and appearance of sulfur (from sulfate in heparin) confirms PEM deposition.

FIG. 5 shows high-resolution X-ray photoelectron spectra of the O1s, N1s and C1s envelopes of cortical bone before and after TMC-heparin PEM deposition. Differences in the spectra confirm deposition of TMC-heparin PEMs on cortical bone surface.

FIG. 6A-B shows high-resolution X-ray photoelectron spectra of the N is, C1s, and S2p envelopes of cortical bone coated with (FIG. 6A) chitosan FD scaffolds and (FIG. 6B) chitosan NF. Bottom row shows neat FD scaffolds and NF; middle row shows FD scaffolds and NF after ammonium hydroxide neutralization, and confirms removal of residual electrospinning solvent from NF; top row shows PEM-modified FD scaffolds and NF with features characteristic of TMC-heparin PEMs, such as ammonium and sulfate.

FIG. 7 shows luminescence of luciferase-expressing adipose-derived stem cells (ASCs) seeded onto murine allografts modified with PEM only, FD and PEM, or NF and PEM, or left uncoated (normalized to the day 1 value for each sample). ASCs proliferated on all four types of allografts. Allografts modified with NF and PEM exhibited the highest luminescence after 21 days. Uncertainties are standard errors of the mean. An asterisk (*) indicates statistically significant differences when compared to the control treatment on the same day (p<0.05, n=4 to 10).

FIG. 8A-C shows scanning electron micrographs showing examples of ASCs cultured on (FIG. 8A) PEM-coated bone, (FIG. 8B) FD and PEM on bone, and (FIG. 8C) NF and PEM scaffolds on bone.

FIG. 9 shows binding of FGF-2 and TGF-β1 to PEM-modified nanofibers confirmed through XPS spectral analysis. FGF-2 amine and amide groups make a significant contribution to the N1s envelope. The appearance of a disulfide bond at 162.7 eV confirmed the presence of TGF-β1.

FIG. 10 shows FGF-2 release from PEM coated allografts observed over 7 days. Only 4% of the total FGF-2 loaded onto the allograft was released over 7 days. The amount of FGF-2 released has been shown to have a mitogenic effect on ovine mesenchymal stem cells.

FIG. 11A-D shows cumulative release of growth factor from bone allografts coated with (FIG. 11A) PEM and FGF-2, (FIG. 11B) FD scaffold and PEM and FGF-2, (FIG. 11C) NF and PEM and FGF-2, and (FIG. 11D) NF and PEM and TGF-β.

FIG. 12 shows a western blot demonstrating Luc-ASCs seeded onto allografts coated with FD and PEM or NF and PEM expressed Alkaline Phosphatase (ALP) and Receptor Activator of NF-κB Ligand (RANKL) at both days 7 and 21, indicating an osteoprogenitor phenotype.

### DETAILED DESCRIPTION

The disclosure relates to nanostructured surface coatings for bone that can serve as a biomimetic periosteum and have an osteoconductive structure and osteoinductive biochemistry. The osteoinductive properties are imparted by the constituent polysaccharides, and the osteoconductivity arises from the nano- and micro-scale structure of the coating.

The following definitions are useful in aiding the skilled practitioner in understanding the disclosure. Unless otherwise defined herein, scientific and technical terms used in the present disclosure shall have the meanings that are commonly understood by those of ordinary skill in the art.

The compositions and methods of the disclosure can comprise, consist essentially of, or consist of, the essential components, as well as optional ingredients described herein.

The terms “porous polysaccharide scaffold” and “porous scaffold-forming polysaccharide” refer to an open framework structure made from polymeric carbohydrate molecules that are adherent to a surface, and to a polysaccharide used to form such a structure, respectively.

The terms “polysaccharide nanofibers” and “nanofiber-forming polysaccharide” refer to fibers having a diameter less than 1000 nm made from polymeric carbohydrate molecules, and to a polysaccharide used to form such fibers, respectively.

The terms “electrospinning” or “electrospun” refer to methods of using an electric current to form fibers from a liquid.

The term “polyelectrolyte multilayer” or “PEM” refers to a multilayered composition comprising alternating polyanion and polycation layers, for example, as described in Almodovar et al. Biomacromolecules. 11:2629-39. 2010 and Almodovar et al., Biomacromolecules. 12: 2755-2765. 2011, the disclosures of which are incorporated herein by reference. A PEM has a plurality of alternating polyanion and polycation layers associated with the adjacent layer(s), e.g., via ionic interactions or hydrogen bonding and totaling, for example, 4 layers, S layers, 6 layers, 7 layers, 8 layers, 9 layers, 10 layers, 11 layers, 12 layers, 13 layers, 14 layers, 15 layers, or more than 15 layers. The thickness of a PEM may be in the rage of about 1 nm to about 100 μm, for example, about 1 nm to about 50 nm, about 100 nm to about 1 μm, or about 5 nm to about 10 nm. A PEM can be formed by alternately exposing a surface to polycation and polyanion solutions to allow for polyelectrolyte adsorption, washing the surface with acidified water in between adsorption steps.

The term “bone graft” refers to a bone or portion thereof that is harvested from a donor and transplanted into a recipient. The term “autograft” refers to a bone graft wherein the donor and recipient are the same. The term “allograft” refers to a bone graft harvested from a donor and transplanted to a different recipient who is of the same species as the donor. As used herein, “bone graft” also refers to a synthetic bone composition, e.g., an implant or prosthesis, that is transplanted into a recipient.

The disclosure provides strategies to improve bone grafts through the creation of a biomimetic periosteum coating. The coating has a porous osteoconductive structure and provides an ideal environment to promote the highly complex cellular processes of bone healing. The enhanced osteogenic and osteoinductive properties of bone surface coated according to the methods described herein improve host-allograft union and the clinical outcome of bone allograft procedures. The coated bone surfaces of the disclosure promote bone healing, stabilize and deliver therapeutic agents, increase osteointegration, and prevent or delay bone graft failure.

In one aspect, the disclosure provides methods of coating a bone surface comprising contacting the bone with (a) a porous scaffold-forming polysaccharide or polysaccharide mixture and/or a plurality of the same or different polysaccharide nanofibers and (b) a PEM composition. In some embodiments, the method comprises applying the porous scaffold-forming saccharide in a solution, e.g., in an aqueous solution such as foam, and then removing the water or other solvent (e.g., by freeze-drying) to create a porous polysaccharide scaffold structure. The porous scaffold-forming polysaccharide can be applied directly to the bone surface or to a PEM composition.

In some embodiments, the method comprises applying a plurality of the same or different polysaccharide nanofibers directly to the bone surface or to a PEM composition. In some embodiments, the method comprises electrospinning the plurality of polysaccharide nanofibers directly onto the bone surface. Electrospinning is a technique whereby a polymer solution is drawn into a fiber by applying a strong electric field between a spinneret and a grounded collector. In practice, the spinneret is a needle on the end of a syringe, mounted on a syringe pump, and the electric field is generated by a laboratory high-voltage power supply. As the spun polymer solution fiber leaves the tip of the spinneret, the solvent rapidly evaporates, and the precipitated polymer fiber is drawn towards the collector by the electric field. The result is a non-woven porous fiber mat that is tough, flexible, and easy to handle. Electrospinning is generally performed on a conducting surface that can be grounded, e.g., aluminum or another metal surface. Because bone is dielectric, successfully electrospinning nanofibers onto bone surface without grounding is challenging. Additionally, the electrospinning procedure results in nanofibers that are easily dissolved in water and require stabilization.

In related embodiments, the method comprises applying a PEM composition, e.g., using layer-by-layer (LbL) deposition, for example, as described in Volpato et al. Acta Biomaterialia. 8: 1551-9. 2012 and Almodovar et al., Biotechnology and Bioengineering. 110: 609-18. 2013; the disclosures of which are incorporated herein by reference. A PEM composition comprising polyanion and polycation layers can be created from the alternating adsorption of polyanions and polycations from solutions onto a charged surface. The surface charge is inverted at each adsorption step, limiting the layer thickness to a few nanometers by electrostatic repulsion. Charge inversion also prepares the surface for the subsequent oppositely charged layer. The LbL method can be used to form uniform conformal coatings on both flat surfaces and irregularly shaped and porous objects that are ultra-thin (e.g., having thicknesses of tens of nanometers).

According to methods of the present disclosure, the porous scaffold-forming polysaccharide and/or plurality of polysaccharide nanofibers is first deposited onto the bone surface, followed by application of the PEM composition to modify the polysaccharide scaffold and/or plurality of polysaccharide nanofibers, or alternatively, the PEM composition is first applied onto the bone surface, followed by deposition of the porous scaffold-forming polysaccharide and/or plurality of polysaccharide nanofibers onto the PEM composition.

Optionally, the native periosteum is removed from the bone surface prior to applying any of the porous scaffold-forming polysaccharide, plurality of polysaccharide nanofibers, and PEM composition. The periosteum can be removed by any technique known in the art, such as by scraping and washing, followed by devitalization and sterilization of the bone in 70% ethanol (e.g., using sonication), and flash freezing at −70° C. Following removal of the periosteum, bone sections can be modified with 11-phosphonoundecanoic acid (PUA), which binds to the surface of the bone and presents a negative charge suitable for PEM deposition.

In another aspect, the disclosure provides a kit comprising (a) a porous scaffold-forming polysaccharide and/or nanofiber-forming polysaccharide; (b) a PEM-forming composition (i.e., the substituent polyanion and polycation); and (c) instructions for coating a bone surface with (a) and (b). In some embodiments, the kit comprises liquid solutions of the porous scaffold-forming polysaccharide or nanofiber-forming polysaccharide and the PEM polycation and polyanion.

In one aspect, the disclosure provides a coated bone comprising a coating comprising (a) a porous polysaccharide scaffold and/or a plurality of polysaccharide nanofibers; and (b) a PEM composition. In some embodiments, the coating comprises a porous polysaccharide scaffold and a PEM composition. In one aspect, the porous polysaccharide scaffold is formed by coating a bone with an aqueous solution (e.g., a foam) comprising a dissolved polysaccharide and then freeze-drying the aqueous solution to remove the water and create a porous polysaccharide scaffold. In related embodiments, the coating comprises (a) a porous polysaccharide scaffold contacting the bone surface and a PEM composition contacting the porous polysaccharide scaffold and/or (b) a PEM composition contacting the bone surface and a porous polysaccharide scaffold contacting the PEM composition.

In other embodiments, the coating comprises a plurality of the same or different polysaccharide nanofibers and a PEM composition. In some embodiments, the plurality of polysaccharide nanofibers is electrospun directly onto the bone surface. In related embodiments, the coating comprises (a) a plurality of polysaccharide nanofibers contacting the bone surface and a PEM composition contacting the plurality of polysaccharide nanofibers and/or (b) a PEM composition contacting the bone surface and a plurality of polysaccharide nanofibers contacting the PEM composition.

In some embodiments, the porous polysaccharide scaffold and/or plurality of polysaccharide nanofibers is neutralized with a strong base and is water-insoluble, e.g., before addition of the PEM composition. Exemplary strong bases, include, but are not limited to, ammonium hydroxide (e.g., greater than 5 M), calcium hydroxide, sodium hydroxide, and any other base that is soluble in water or a slightly polar solvent such as ethanol. Such bases act to extract any residual acid and neutralize the polysaccharide to render it water-insoluble. Such neutralization of the polysaccharide scaffold or nanofibers stabilizes the structures, e.g., to allow for the subsequent deposition of the PEM composition or successful transplantation.

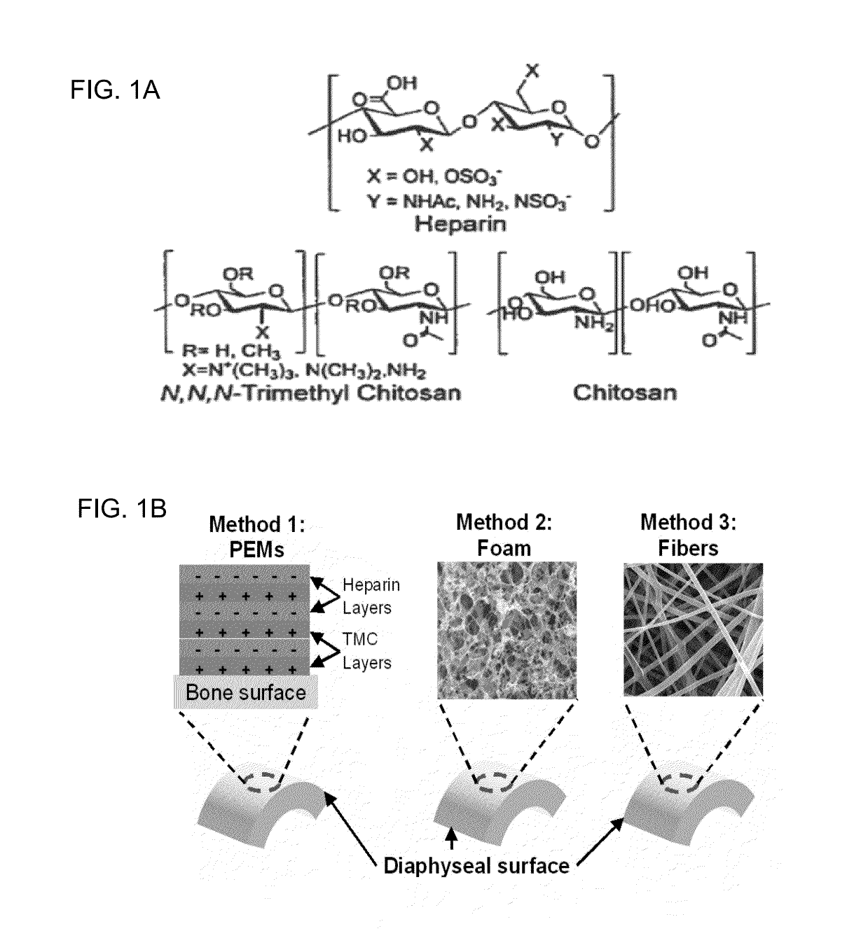

The PEM, polysaccharide scaffold (foam), and polysaccharide nanofibers of the disclosure are illustrated schematically in FIG. 1B. The three layers provide different surface topographies, but similar surface chemistry. The PEM provides coatings that can be less than 10 nanometers thick that conform to the topography of the underlying surface. The freeze-dried foam provides a highly porous scaffold coating, which can swell considerably when in contact with aqueous fluid, and which presents concave surface features with interconnected pores at the micro- and nano-scale. The electrospun nanofibers provide a porous network, which is stable when in contact with an aqueous fluid and presents a convex surface on the micro- and nano-scale, with interconnected pores.

In various embodiments, the porous scaffold-forming polysaccharide, porous polysaccharide scaffold, or plurality of polysaccharide nanofibers comprises a polysaccharide selected from the group consisting of chitosan, acylated (e.g., formylated) chitosan, alkylated (e.g., methylated) chitosan, chitosan modified with another functional group such as an aryl group, or chitosan modified by attachment of a pro-drug or cross-linkable functional group, and combinations thereof. Chitosan, a deacetylated derivative of the naturally abundant polysaccharide chitin, is a material well-suited for bone tissue engineering. Chitosan has been demonstrated to be biocompatible for a number of cell and tissue engineering applications (VandeVord et al. Journal of Biomedical Materials Research. 59:585-90. 2002; Molinaro G, et al. Biomaterials. 23:2717-22. 2002; Shin et al. J Periodontol. 76:1778-84. 2005), is biodegradable (Tomihata et al. Biomaterials. 18:567-75. 1997; Nordtveit et al. Carbohydrate Polymers. 29:163-7. 1996; Vårum et al. Carbohydrate Research. 299:99-101. 1997), has antibacterial activity (Almodovar et al. Biotechnology and Bioengineering. 110(2): 609-618. 2013; Jia et al. Carbohydrate Research. 333:1-6. 2001; Loke et al. J Biomed Mater Res. 53:8-17. 2000), and promotes wound healing (Biagini et al. Biomaterials. 12:281-6. 1991; Ueno et al. Biomaterials. 20:1407-14. 1999; Azad et al. Journal of Biomedical Materials Research Part B: Applied Biomaterials. 69B:216-22. 2004). Furthermore, chitosan can be readily processed into various tissue engineering scaffolds and surface coatings (Almodovar et al. Biotechnology and Bioengineering. 2013, supra; Costa-Pinto et al., Biomacromolecules. 10: 2067-73. 2009). In some embodiments, the polysaccharide is chitosan modified prior to formation of the coating. In other embodiments, the polysaccharide is chitosan modified following application of the polysaccharide scaffold/nanofibers and PEM composition to form a coated bone.

In various embodiments of the disclosure, the PEM composition comprises a polyanion selected from the group consisting of a glycosaminoglycan (GAG), an anionic polysaccharide, a polymer, and combinations thereof. In some embodiments, the PEM composition comprises a GAG selected from the group consisting of heparin, heparin sulfate, hyaluronan, chondroitin sulfate, dermatan sulfate, keratan sulfate, and combinations thereof. GAGs are important components of skeletal tissues with biochemical and biophysical functions. In particular, sulfated GAGs, such as heparin, heparan sulfate, and chondroitin sulfate, bind and stabilize growth factors in the extra- and peri-cellular space. GAGs serve as a reservoir for stabilized growth factors, and they potentiate the binding of growth factors to the cell surface receptors (Boddohi et al. Adv Mater. 22:2298-3016, 2010). Binding sequences for FGF-2 in sulfated GAGs are believed to promote dimerization or oligomerization of the protein along the GAG chain, and thereby activate the mitogenic activity of FGF-2 (Zamora et al. Bioconjugate Chem. 13:920-6. 2002; Berry et al. FASEB J. 15:1422. 2001; Guimond et al. J Biol. Chem. 268:23906-14. 1993). GAGs have also been used to stabilize and deliver TGF-β proteins.

In some embodiments, the PEM composition comprises an anionic polysaccharide or other polyanion selected from the group consisting of sulfated chitosan, sulfated dextran, a fucan, a carrageenan, a pectin, poly(styrene sulfonate) and combinations thereof. The anionic polysaccharide or other polyanion can serve as a “GAG-mimic” having functional similarity to a GAG in terms of binding and stabilizing growth factors. Other “GAG-mimics” suitable for use in the PEM composition include polysaccharides comprising disaccharide repeating units comprising a hexuronic acid unit and a hexosamine unit.

In some embodiments, the PEM composition comprises a polycation selected from the group consisting of chitosan, acylated chitosan, alkylated chitosan (e.g., a methylated chitosan such as TMC), poly-lysine, polyethyleneimine, and combinations thereof. Chitosan is structurally similar to the GAGs, but behaves as a polycation, rather than a polyanion. The free amino group in chitosan has a pKₐ of about 6.5, giving chitosan its cationic behavior, which can be used to interact with a polyanion.

In some embodiments, the PEM composition comprises heparin and N,N,N-trimethylchitosan.

In some embodiments, the coating further comprises a therapeutic agent, e.g., adsorbed to the polysaccharide scaffold, polysaccharide nanofibers, and/or PEM composition. In some embodiments, the therapeutic agent is selected from the group consisting of a growth factor, a progenitor cell, a hormone, an anti-inflammatory agent, an antibiotic, a polynucleotide, a chemotherapeutic agent and combinations thereof. Accordingly, the disclosure provides methods of stabilizing and delivering a therapeutic agent comprising administering a coated bone described herein to a subject in need thereof and use of the coated bone as a medicament.

Exemplary growth factors include, but are not limited to, bone morphogenetic proteins (BMPs), epidermal growth factor (EGF), fibroblast growth factor (FGF), insulin-like growth factor (IGF), platelet-derived growth factor (PDGF), transforming growth factor (TGF) alpha and beta, tumor necrosis factor (TNF), and vascular endothelial growth factor (VEGF). Although growth factors have significant therapeutic potential, they are also particularly unstable under in vivo conditions and have very short half-lives. The plasma half-life of FGF-2 and TGF-β1 have been reported to be 1.5 minutes and 11 to 160 minutes, respectively (Tessmar et al. Adv Drug Deliver Rev. 59:274-91. 2007). Such extremely short half-lives undermine the direct delivery of growth factors to injury sites as a possible treatment option, unless extremely high doses are used. In some embodiments, the disclosure provides a coated bone comprising a coating comprising a growth factor bound to a GAG or anionic polysaccharide. The binding of the growth factor to the GAG or GAG-mimic stabilizes the growth factor and promote growth factor signaling by presenting the growth factor to cells in a context that mimics the biological presentation in the extracellular matrix.

Exemplary progenitor cells include, but are not limited to, mesenchymal stem cells, neural stem cells, embryonic stem cells, adipose-derived mesenchymal stem cells, embryonic fibroblasts, bone marrow stem cells, skin stem cells, and umbilical cord blood stem cells. Exemplary hormones include, but are not limited to, melatonin, serotonin, thyroxin, epinephrine, norepinephrine, dopamine, adiponectin, adrenocorticotropic hormone, angiotensinogen, antidiuretic hormone, atrial natriuretic peptide, calcitonin, cholecystokinin, corticotrophin-releasing hormone, erythropoietin, follicle-stimulating hormone, gastrin, ghrelin, glucagon, growth hormone-releasing hormone, growth hormone, insulin, insulin-like growth factor, leptin, luteinizing hormone, orexin, oxytocin, parathyroid hormone, secretin, aldosterone, testosterone, estradiol, progesterone, lipotropin, brain natriuretic peptide, histamine, endothelin, and enkephalin. Exemplary anti-inflammatory agents include, but are not limited to, corticosteroids and non-steroidal anti-inflammatory drugs (NSAIDS). Exemplary antibiotics include, but are not limited to, antibacterial agents, antimycotic agents, antifungal agents, antimicrobial agents, and antiviral agents.

Exemplary chemotherapeutic agents include, but are not limited to, alkylating agents, antibiotics, antimetabolites, differentiating agents, mitotic inhibitors, steroids, topoisomerase inhibitors, and tyrosine kinase inhibitors, such as azacitidine, axathioprine, bevacizumab, bleomycin, capecitabine, carboplatin, chlorabucil, cisplatin, cyclophosphamide, cytarabine, daunorubicin, docetaxel, doxifluridine, doxorubicin, epirubicin, etoposide, fluorouracil, gemcitabine, herceptin, idarubicin, mechlorethamine, melphalan, mercaptopurine, methotrexate, mitoxantrone, oxaliplatin, paclitaxel, tafluposide, teniposide, tioguanine, retinoic acid, valrubicin, vinblastine, vincristine, vindesine, vinorelbine, and combinations thereof. Exemplary polynucleotides include, but are not limited to, single- and double-stranded DNA and RNA, e.g., for use in gene therapy.

In some embodiments, the coated bones of the disclosure prevent the degradation of the therapeutic agent. In some embodiments, the coated bones may be used to deliver an effective amount of the therapeutic agent to a subject in need thereof, which is an amount effective to achieve a desired biological, e.g., clinical, effect. An effective amount of a therapeutic agent varies with the nature of the disease being treated, the length of time that activity is desired, and the age and the condition of the subject. In some embodiments, the therapeutic agent is released over a prolonged period, for example, for at least one week, at least two weeks, at least three weeks, at least one month, at least two months, at least three months, at least six months, or longer than six months.

In various embodiments, a coated bone of the present disclosure is a bone graft. In some embodiments, the bone graft is selected from the group consisting of an autograft, an allograft, a xenograft, and a prosthesis. In some embodiments, the bone graft is an allograft, for example, an allograft wherein the periosteum is removed before the bone surface is coated according to the methods of the disclosure. The disclosure thus also provides methods of transplanting bone comprising administering the bone graft described herein to a subject in need thereof.

The following numbered embodiments each define one or more exemplary embodiments of the disclosure:

1. A polyelectrolyte multilayer applied to allograft bone surfaces, where the polyelectrolyte multilayer contains glycosaminoglycans (heparin, heparan sulfate, hyaluronan, chondroitin sulfate, dermatan sulfate, keratan sulfate, etc.) as the polyanion, or another polysaccharide with sulfate substituents, such as sulfated chitosan or sulfated dextran as the polyanion. The surface of the bone might first be cleaned to remove the periosteum, and chemically modified with an agent, such as a phosphonic acid, that permits the polyelectrolyte multilayer to bind to the bone. The agent might be 11-phosphonoundecanoic acid (PUA), which binds to the surface of the bone and presents a negative charge, suitable for polyelectrolyte multilayer deposition.

2. A coating on allograft bone surfaces made by freeze-drying a porous polymer scaffold onto the bone, where the porous polymer scaffold is a polysaccharide such as chitosan or a chitosan derivative (e.g. methylated chitosan).

3. A coating on allograft bone surfaces made by freeze-drying a porous polymer scaffold onto the bone, and subsequently modifying the porous polymer scaffold with polyelectrolyte multilayers containing sulfated polysaccharides as in embodiment 1.

4. A coating on allograft bone surfaces made by directly electrospinning polymeric nanofibers onto the bone surface.

5. A coating on allograft bone surfaces made by directly electrospinning polymeric nanofibers onto the bone surface, and subsequently modifying the porous polymer scaffold with polyelectrolyte multilayers containing sulfated polysaccharides as in embodiment 1.

6. Coatings as in any of embodiments 1, 3, and 5 where the sulfated polysaccharides in the polyelectrolyte multilayer are used to stabilize and deliver growth factors that promote healing, such as members of the fibroblast growth factor (FGF) family and the transforming growth factor beta superfamily (TGF-β).

7. Coatings as in any of embodiments 1, 3, and 5 where the sulfated polysaccharides in the polyelectrolyte multilayer are used to stabilize and deliver other heparin-binding signaling proteins, such as parathyroid hormone (PTH) and vascular endothelial growth factor (VEGF).

8. Coatings as in any of embodiments 2, 3, 4, 5, 6 and 7 where the freeze-dried polymer scaffold or nanofiber network provide an osteoconductive structure.

9. Coatings as in embodiment 8 where the osteoconductive structure is used to deliver progenitor cells, such as bone marrow stromal cells, stem cells from bone marrow, or stem cells from adipose tissue.

10. Coatings as in embodiment 8 where the polyelectrolyte multilayers, freeze-dried scaffolds, and/or nanofibers contain agents with antibacterial, antimycotic, antifungal, and/or antimicrobial.

11. Applications of the coatings described in any of embodiments 1-10 above in human and mammal bone allograft procedures.

12. Preparation of a porous chitosan foam coating on the bone allograft surfaces by freeze drying chitosan onto the bone.

13. Preparation of nanofiber chitosan scaffolds by electrospinning chitosan directly onto the bone. This is the first demonstration of the direct modification of bone with a polymer nanofiber.

14. Method for stabilizing the porous freeze-dried and nanofiber scaffolds by treatment with ammonium hydroxide. This stabilization is essential to make the scaffolds durable with respect to multiple aqueous processing techniques required to modify them with polyelectrolyte multilayers and growth factors.

15. Modification of stabilized porous freeze-dried and nanofiber scaffolds with polyelectrolyte multilayers, maintaining their micro and nanostructure.

16. Modification of stabilized porous freeze-dried and nanofiber scaffolds with polyelectrolyte multilayers and growth factors, maintaining the growth factor activity.

17. Fabrication of custom electrospinning nanofiber collection apparatus to collect the nanofibers on the bone surface.

18. Delivery of growth factors such as FGF-2 from polyelectrolyte multilayers.

The present disclosure will be more readily understood by reference to the following Examples, which are provided by way of illustration and are not intended to be limiting.

### Example 1

### Preparation and In Vitro Analysis of Bone Coatings

Materials and Methods

Chitosan (80 kDa, 5% acetylated confirmed through ¹H NMR) was acquired from Novamatrix (Sandvika, Norway). Heparin sodium from porcine intestinal mucosa (14.4 kDa, 12.5% sulfur) was purchased from Celsus Laboratories (Cincinnati, Ohio). Chitosan was methylated to make TMC following a previously reported method (de Britto at al. Carbohydrate Polymers. 69: 305-10. 2007). Aqueous solutions were made by dissolving heparin or TMC in water at 0.01 M solutions (based on a saccharide unit basis). The structures of these polysaccharides are shown in FIG. 1A. PUA (11-phosphonoundecanoic acid), glutaraldehyde, and sucrose were obtained from Sigma-Aldrich (St. Louis, Mo.). Hexamethyldisilazane was purchased from Alfa Aesar (Ward Hill, Mass.). Sodium cacodylate trihydrate was purchased from Polysciences Inc (Warrington, Pa.). Dimethyl sulfoxide was purchased from EMD Chemicals Inc. (Gibbstown, N.J.). Dichloromethane (DCM) and trifluoroacetic acid (TFA) were purchased from Acros Organics (New Jersey, US). Aqueous solutions were made using ultrapure water (18.2 MΩ·cm water from a Millipore Synthesis water purification unit). PVDF 0.22 μm filters and phosphate buffered saline (PBS) were obtained from Fisher-Scientific (Pittsburgh, Pa.). Dulbecco's Modification of Eagle's Medium-low glucose, MEM vitamins, MEM nonessential amino acids, antibiotic-antimycotic solution were obtained from Corning Cellgro (Manassas, Va.). Fetal bovine serum was obtained from Atlas Biologics (Fort Collins, Colo.). Recombinant human fibroblast growth factor 2 (rhFGF-2), recombinant human transforming growth factor-β1 (rhTGF-β1), and Human FGF basic Quantikine ELISA kit were purchased from R&D Systems (Minneapolis, Minn.).

Bone Tissue Harvest and Cleaning:

Murine femurs and humeri allografts (4 mm) were harvested from C3H mice (age 7-9 weeks) sacrificed for another study. The allografts were rinsed with saline and frozen at −70° C. for a minimum of 2 weeks. They were then thawed and rinsed with ultrapure water. The allografts were cleansed by removal of residual bone marrow from the intramedullary cavity, mechanically scraped with a razor to remove any remaining soft tissue, and then sonicated with 70% ethanol for 3 hours and dried under vacuum.

Luciferase Expressing ASC Stem Cell Isolation and Expansion:

Luciferase-expressing ASCs were isolated from abdominal adipose tissue of (FVB/NTsv-Tg(svyb-luc)-Xen) mice from Taconic (Hudson, N.Y.). Adipose tissue underwent a collagenase digestion for 30 minutes. ASCs were then plated for 24 hours, and plastic-adherent cells were selected by rinsing and aspirating to remove non-adherent cells. ASCs were expanded to passage 3.

Cortical Bone Allografts Coatings:

Allografts diaphyseal surfaces were coated with one of three tissue engineering scaffolds—polyelectrolyte multilayers (PEMs), freeze dried chitosan (FD), and electrospun chitosan nanofibers (NF). In order to adhere the PEMs to the bone surface, allografts were first treated with a self-assembled monolayer of PUA deposited using a 10 mM solution of PUA in dimethyl sulfoxide. The bones were immersed in this solution overnight. The treated allografts were then subjected to layer-by-layer (LbL) deposition of alternating solutions of TMC and heparin with TMC being the first layer deposited. TMC and heparin solutions were made by dissolving TMC and heparin at a 0.01 M concentration on a per saccharide basis in ultrapure water. The solutions were filtered with a 0.22 μm PVDF filter. Bone allografts were placed in a 48-well plate and subjected to an initial 5 minute rinse with ultrapure water. The rinse water was aspirated and the appropriate PEM solution was pipetted into each well plate containing each bone allograft. Five minute adsorption steps were used for each polyelectrolyte solution with a 5-minute rinse step with ultrapure water between PEM adsorption steps. All steps were performed under gentle agitation using a Barnstead Labline titer plate shaker 4625 (Dubuque, Iowa). Six-layer PEMs were deposited directly on the allograft surface resulting in a terminal heparin layer.

To create a porous chitosan scaffold, allograft bone was cast in a custom cylindrical mold with a 6% (w/w) chitosan in 0.34 M acetic acid solution and frozen at −20° C. for 24 hours. The custom mold assembly was then subsequently lyophilized for 48 hours, after which the chitosan scaffold on the allograft was mechanically shaved with a razor and neutralized with a 5 M NH₄OH solution for 6 hours to form the FD scaffold. After neutralization, the FD scaffold was ready for surface modification with TMC and heparin PEMs as described below.

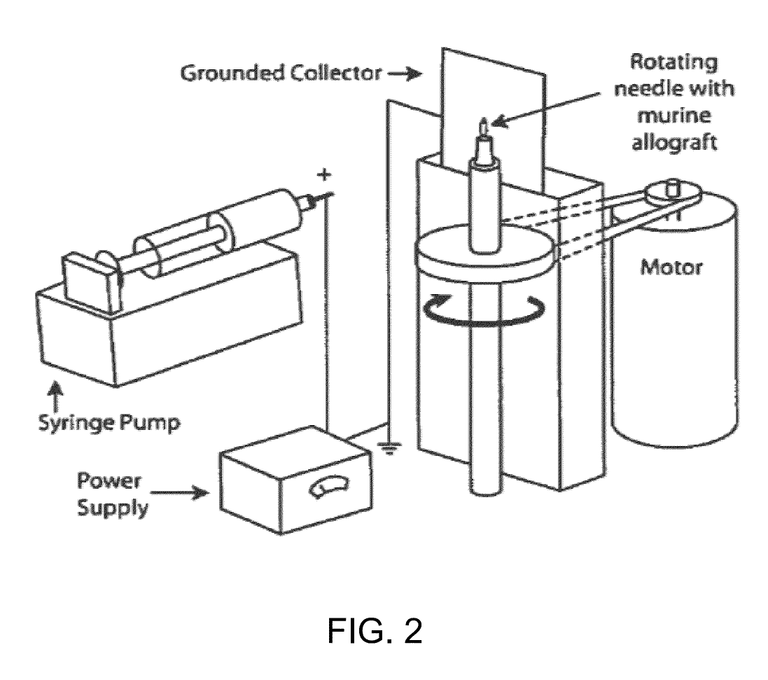

To create a porous chitosan structure with an alternative structure, chitosan nanofibers were directly electrospun onto the bone diaphyseal surface using a custom rotating collector apparatus, as seen in FIG. 2. A 1/16-inch copper plate covered with grounded aluminum foil served as a collection plate. A rotating shaft with a custom allograft holder was placed in front of the grounded plate. A syringe pump containing a glass syringe and 18-gauge blunt-tip needle was placed across from the grounded collector. The needle tip-to-collector distance was 7 inches. Chitosan was dissolved as a 7% (w/v) solution in a 7:3 TFA:DCM ratio for 24 hours before electrospinning. The chitosan solution was supplied at a volumetric flow rate of 1 mL/hr using a Kent Scientific Genie Plus syringe pump (Torrington, Conn.). The solution was electrospun at 18 kV using a high voltage DC power supply (Gama High Voltage Research Ormond Beach, Fla.). The nanofibers were then stabilized by neutralizing in a 5 M NH4OH solution for 6 hours, as has been previously reported (Almodovar et al. Macromol Biosci. 11:72-6. 2011, incorporated herein by reference) to form the NF coating. After neutralization, the NF coating was ready for subsequent surface modification with TMC and heparin PEMs as described below.

Surface Modification of FD and NF Scaffolds with PEMs:

The chitosan NF and FD scaffold-coated allografts were subjected to LbL deposition of TMC and heparin as following the same procedure described above for modifying the bone surfaces with PEMs. However, for the NF and FD scaffolds, heparin was used as the initial layer. Seven layers were deposited to achieve a terminal heparin layer.

Growth Factor Binding and Release:

Diaphyseal allografts of murine humeri and femurs (4-5 mm sections) were soaked in a “high” concentration (1000 ng/mL) of growth factor, or a “low” concentration (500 ng/mL or 100 ng/mL) of growth factor. The amounts of growth factor bound to the allografts (average of three allografts per condition) are shown in Table 1. When either FGF-2 or TGF-β is bound to PEM-coated, FD-coated, or NF-coated bones, modified with TMC-heparin PEMs, the total amount of growth factor bound can be varied from several nanograms to hundreds of nanograms.

TABLE 1
Average amounts of growth factor bound to allografts
with different coatings, for both the “high”
and “low” growth factor concentrations

| Coating type | FGF-2 loaded (ng) | TGF- β loaded (ng) |
|---|---|---|
| PEM | 7.4 ± 0.7 | 269 ± 8 |
| | High | High |
| | 3.2 ± 0.9 | 20.9 ± 3 |
| | Low | Low |
| Scaffold/PEM | 312 ± 4 | — |
| | High | |
| | 11.4 ± 0.1 | — |
| | Low | |
| Nanofibers/PEM | 55 ± 2 | 34 |
| | High | High |
| | 6.5 ± 0.4 | 18 ± 10 |
| | Low | Low |

rhFGF-2 and rhTGF-β1 Binding to PEM Coated Allografts, PEM Modified FD and PEM Modified NF Scaffolds for XPS Analysis:

PEM coated allografts, PEM modified FD and PEM modified NF scaffolds on allografts were prepared using the procedures mentioned above. rhFGF-2 and rhTGF-β1 were reconstituted per manufacturer's protocol. Solutions of 100 ng ml⁻¹ of each growth factor were made and PEM coated allografts, PEM-modified FD and PEM-modified NF scaffolds on allografts were immersed in 0.5 ml of rhFGF-2 or rhTGF-β1 solutions in 48-well plates. The allografts and growth factor containing solutions were gently agitated for 1 hour using a plate shaker to allow for growth factor binding onto the PEMs.

rhFGF-2 Kinetic Release Assay from PEM Coated Allografts: rhFGF-2 was bound to PEM coated allografts in triplicate using 0.5 ml of a 1000 ng ml⁻¹ rhFGF-2 solution and 1 hour of gentle agitation in a 48-well plate using a plate shaker. After adsorption of rhFGF-2, the rhFGF-2 loading solution was collected and the allografts were transferred to new wells in the 48-well plate. Allografts with bound rhFGF-2 were then immersed in 500 μl of PBS and incubated at 37° C. and 5% CO₂. Aliquots of 150 μl of PBS were removed from the wells containing allografts and replenished with fresh PBS at predetermined time points. Aliquots were taken at the following time points: 0, 0.15, 0.30, 1, 3, and 7 days. The collected rhFGF-2 loading solution and rhFGF-2 containing aliquots were frozen at −20° C. for further analysis with ELISA. R&D System's Human FGF basic Quantikine ELISA kit was used to assess rhFGF-2 concentrations in rhFGF-2 loading solution and sample aliquots according to the manufacturer's kit instructions. rhFGF-2 containing samples were serially diluted in order to ensure samples produced a detectable signal within the ELISA kit's standard curve.

Macroscopic Characterization:

PEM-modified and NF-modified allografts were coated with 10 nm of gold and FD-modified allografts were coated with 20 nm of gold before imaging with a scanning electron microscope. Micrographs were taken of the unmodified bone surface, unmodified scaffolds on bone, the scaffolds' intermediate processing step, and scaffolds after PEM deposition.

X-Ray Photoelectron Spectroscopy (XPS) of Surface Modified Scaffolds:

Surface chemistry of the modified bones was obtained using a Phi Electronics 5800 Spectrometer (Chanhassen, Minn.). Spectra were obtained with a monochromatic Al Kαx-ray source (hv=1486.6 eV), a hemispherical analyzer, and multichannel detector. High resolution spectra were obtained using a 23.5 eV analyzer pass energy with 0.1 eV steps and an X-ray spot of 800 μm. All spectra were obtained with a photoelectron takeoff angle of 45°. A low energy electron gun was used for charge neutralization. Spectra curve fitting was done using Phi Electronics Multipak version 9.3 (Chanhassen, Minn.). Curve fitting of all spectra used a Shirley background. Gaussian peaks were fit according to expected functional groups. The height of each peak was fit first while keeping each peaks' position, full width half max (fwhm), and percent Gaussian fixed. Then the fwhm, percent Gaussian, and finally position were fit while minimizing the chi squared value.

Luciferase-Expressing Adipose-Derived (ASCs) Stem Cell In-Vitro Response:

C3H allografts were prepared in triplicate with each of the three scaffolds (PEM, FD, and NF) described above. FD and NF scaffolds were subsequently modified with heparin-terminated TMC-heparin PEMs as described above. Each allograft was seeded at a concentration of 100,000 cells mL⁻¹ in a 48-well plate and cultured in ASC maintenance media. ASCs adhered to tissue culture polystyrene wells were used as a positive control and were seeded in triplicate at 10,000 cells mL⁻¹ ASCs were allowed to attach to their substrate for 24 hours and then the allografts were transferred to new wells. The ASCs were then cultured for 13 days with media changes every 2 to 3 days. At days 1, 4, and 13, firefly luciferin substrate was added to each well plate at a concentration of 50 μg mL⁻¹, incubated for approximately 5 minutes at room temperature, and then bioluminescent readings were taken on an IVIS in vivo imaging system from PerkinElmer (Waltham, Mass.) using a humidified chamber. Images were thresholded to eliminate background signal and the total photon flux was calculated for each picture. The average total flux of each scaffold was calculated and then normalized to each scaffolds' day 1 reading. After 13 days of culture, ASC-seeded allografts were fixed using a 2% glutaraldehyde solution prepared in 0.2 M sodium cacodylate and 0.1 M sucrose buffer solution. The ASC seeded allografts were then dehydrated using an increasing concentration ethanol series. Allografts were then imaged after being sputter coated with gold as mentioned above.

Luc-ASCs Phenotype Evaluation by Western Blotting:

Luciferase-expressing ASCs (Luc-ASCs) cultured on modified allografts were evaluated for osteogenic differentiation after 7 and 21 days of in-vitro culture. Samples were rinsed twice in cold Hank's Balanced Salt Solution (HBSS) before being lysed in a commercial RadiolmmunoPrecipitation Assay (RIPA) buffer obtained from Thermo-Scientific (Rockford, Ill.) containing 3× protease inhibitors. Samples were lysed using a handheld sonicator wand while keeping samples on ice. Replicate lysates were pooled together and centrifuged for 15 minutes at 4° C. to pellet cell debris and the supernatant was collected and frozen until ready to be further assayed. Samples were thawed and then denatured and reduced before running on a 4-20% Ready Gel Tris-HCl gel (Bio-rad, Hercules, Calif.) using a Bio-rad Mini-Protean 3 electrophoresis unit. Proteins were transferred onto an Immobilon-PSQ PVDF membrane (EMD Millipore, Billerica, Mass.) using a wet tank transfer method for 2 hours at 4° C. Membranes were blocked in 5% non-fat milk for 1 hour at room temperature, then rinsed three times for 5 minutes each. Blots were probed initially for ALP using anti-alkaline phosphatase primary antibody (1:10,000, ab108337, Abcam, Cambridge, Mass.) overnight at 4° C. Horseradish peroxidase-conjugated Goat anti-Rabbit IgG H&L secondary antibody (1:20,000 Abcam ab6721) was used, and membranes were developed using enhanced chemiluminescence substrate solution (Thermo-Scientific, Rockford, Ill.). Immunoblots were imaged using a Bio-rad Chemidoc XRS+ imager (Hercules, Calif.). Immunoblots were subsequently stripped with Thermo Restore stripping buffer (Thermo-Scientific, Rockford, Ill.) and reprobed for osteocalcin (1:3000, Millipore ab10911), osteonectin (0.4 μg/ml, Abcam ab55847), osteopontin (0.1 μg/ml, Abcam ab11503) and RANKL (1:5000, Abcam ab124797) and developed. Blots were stripped and blocked in between probings.

Statistical Analysis:

Bioluminescence measurements on days 4 and 13 were analyzed using ANOVA. Comparisons between groups were performed using a Tukey's post hoc test. A p value less than 0.05 was considered significant. Minitab 16 software was used for the analysis.

### Results

Macroscopic Analysis:

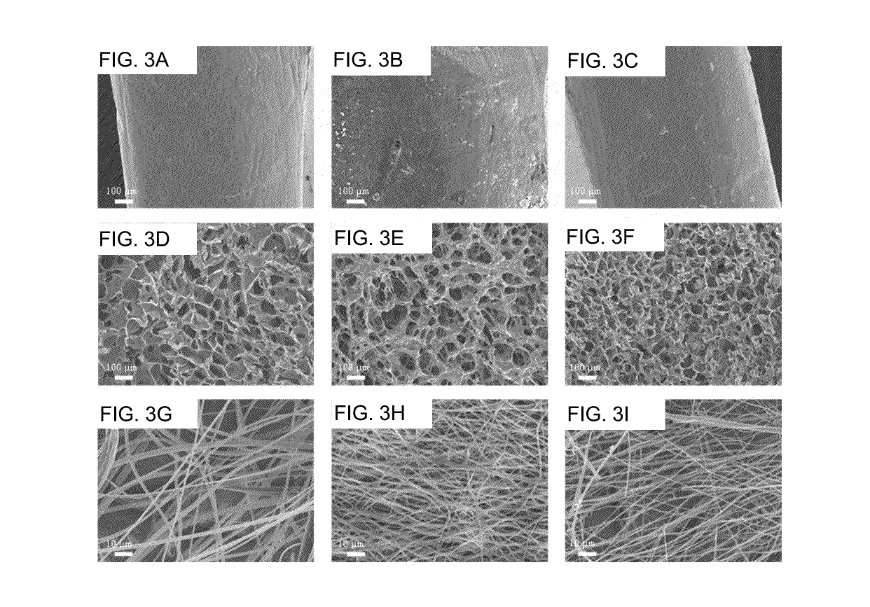

Scanning electron micrographs revealed successful allograft diaphyseal surface coatings on the entire allograft with both the chitosan freeze-dried (FD) scaffold and the chitosan electrospun nanofibers (NF) as seen in FIG. 3A-I. These polysaccharides were stable with respect to further aqueous modification steps (neutralization with ammonium hydroxide and LbL deposition of seven alternating layers of TMC and heparin) with no gross morphological changes, as evidenced by FIG. 3U, FIG. 3F, and FIG. 3I. Allografts directly coated with only PEMs of TMC and heparin exhibited minimal surface topographical changes, as was expected, since the PEMs should have a thickness of approximately 10 to 15 nm, which would be indistinguishable in the micrographs.

XPS Analysis of Surface Modified Allografts:

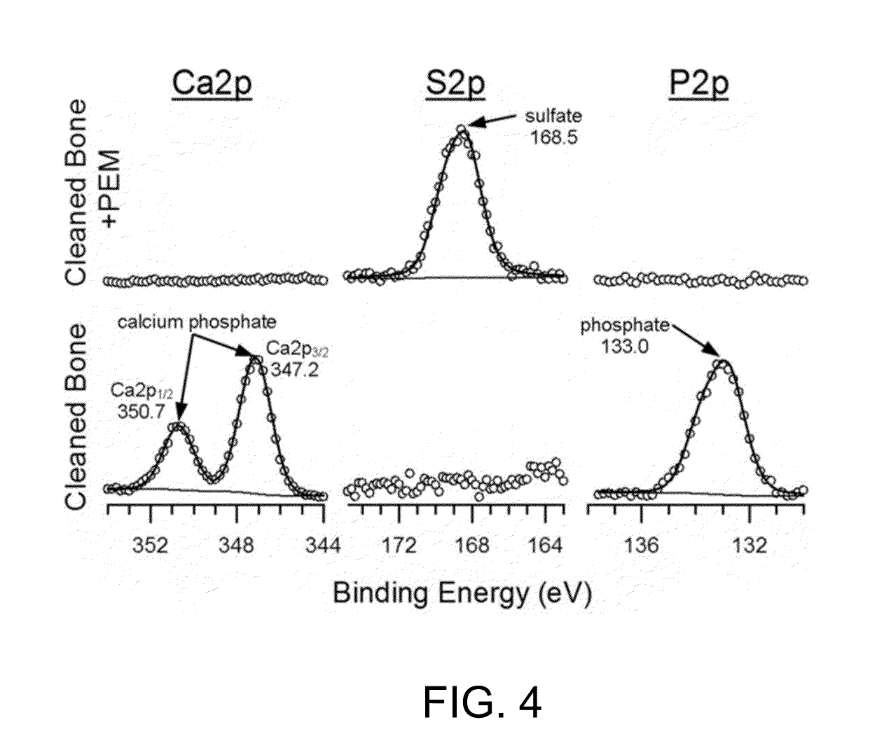

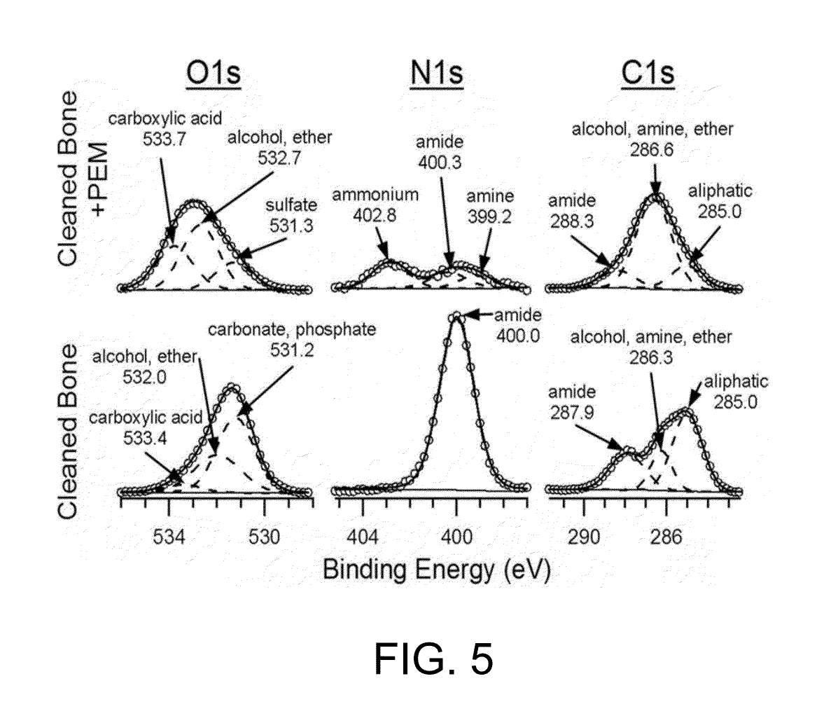

The surface chemistry of PEM-modified bone and scaffolds was characterized using survey and high-resolution XPS spectra. High-resolution spectra confirmed deposition of TMC and heparin on the allograft diaphyseal surface. FIG. 4 shows complete attenuation of the Ca2p and P2p envelopes indicating complete surface coverage with PEMs. FIG. 4 also shows the appearance of a sulfur S2p peak at 168.5 eV (sulfate), which confirmed heparin deposition within the heparin-terminated PEMs. In FIG. 5, the attenuation of the amide peak at 400 eV and the appearance of an ammonium and amine peak in the N1s envelope at 402.8 eV and 399.2 eV, respectively, confirmed LbL deposition of the TMC-heparin PEM. Changes in the C1s and O1s envelopes of the XPS spectra were also characteristic of polysaccharide-based PEM deposition on bone.

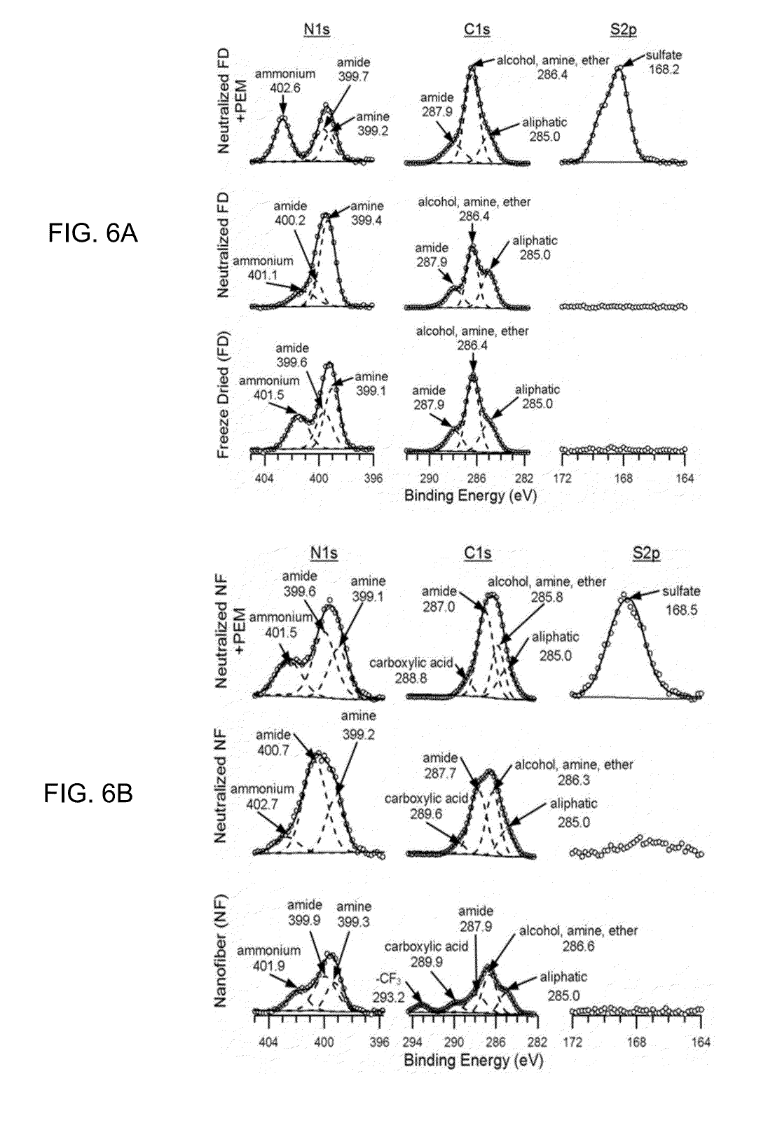

FIG. 6A-B shows the N1s, C1s and S2p envelopes for the FD (FIG. 6A) and NF (FIG. 6B) modifications of bone. The bottom row is the neat FD and NF immediately after freeze drying or electrospinning. The middle row is the FD and NF after ammonium hydroxide neutralization, and the top row is the PEM-modified FD and NF. The neat FD and NF had significant contributions from the trifluoroacetate at 289.9 and 293.2 eV in the C1s envelope, indicating residual solvent from the electrospinning, which must have been in the form of a salt with the amine groups in the electrospun chitosan, as the trifluoroacetic acid was not removed under the high vacuum of the XPS chamber. As was reported previously, neutralization with ammonium hydroxide completely removed the trifluoracetate from the nanofibers (Almodovar et al. Macromol Biosci. 2011, supra). LbL addition of the PEM to the FD and NF scaffolds resulted in similar characteristics in the N1s, C1s and S2p envelopes, such as addition of sulfate and ammonium.

Luc-ASCs Stem Cell In-Vitro Response:

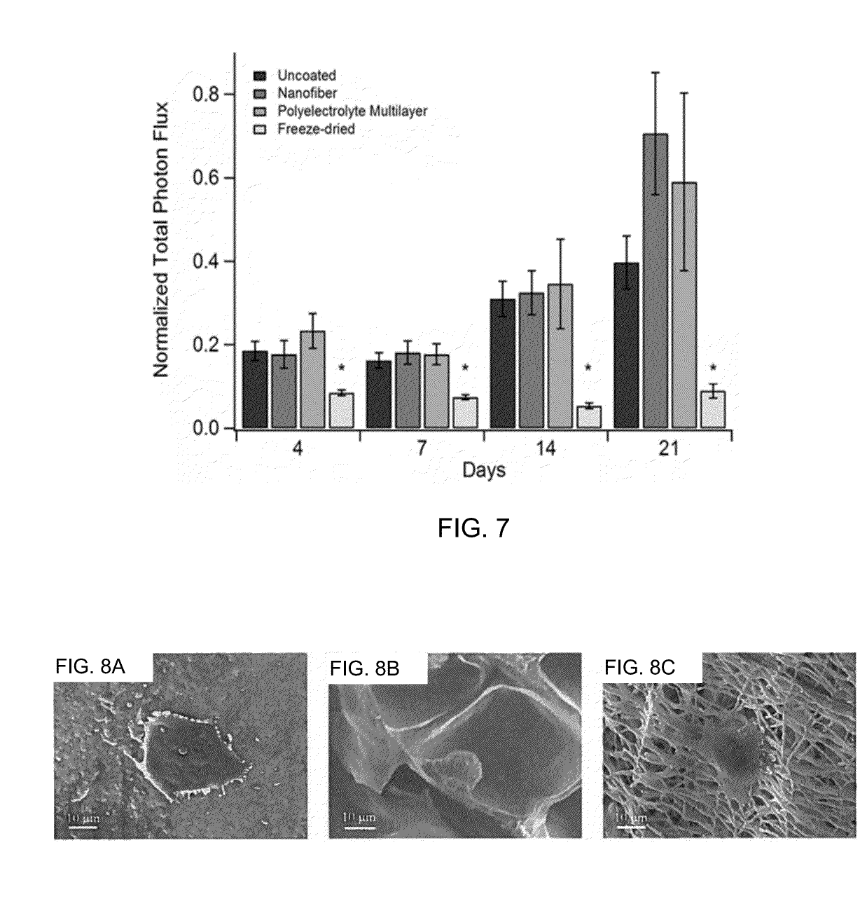

Luc-ASCs were seeded onto each of the three tissue engineering scaffolds to discern whether they might be used to support ASC transplantation. FIG. 7 shows the normalized average photon flux after up to 21 days of in vitro culture. Values were normalized to the photon flux on day 1 for each sample. Proliferation was observed for ASCs on all control, PEM-coated, FD and PEM-coated, and NF and PEM-coated allografts. Firefly luciferin uniquely requires ATP as a co-factor in order to actively bioluminesce. The presence of ATP activity in ASCs demonstrated cellular metabolic activity and indicated viable cells. The NF and PEM-coated allografts exhibited the largest increase in bioluminescent flux, indicating the greatest ASC proliferation, which could be explained by the ECM-mimetic structure and porosity of the coating. FD and PEM-coated allografts were the only allografts to exhibit a significant difference in normalized average photon flux compared to all other treatments at each timepoint (p<0.05). FIG. 8A-C shows ASCs adopted a flat cellular morphology on PEM-coated (FIG. 8A) and NF and PEM-coated (FIG. 8C) allografts while ASCs on FD and PEM-coated allografts (FIG. 8B) adopted a spherical morphology. ASC survival up to 21 days indicated that PEM, FD and PEM, and NF and PEM scaffolds possessed cytocompatibility characteristics conducive to bone tissue engineering applications. The scanning electron micrographs demonstrated minimal degradation of both FD and NF coatings on allografts throughout the various aqueous processing steps (FIG. 3A-I) and after a 13-day incubation in cell media (FIG. 8A-C).

Growth Factor Binding Characterization and Kinetic Release:

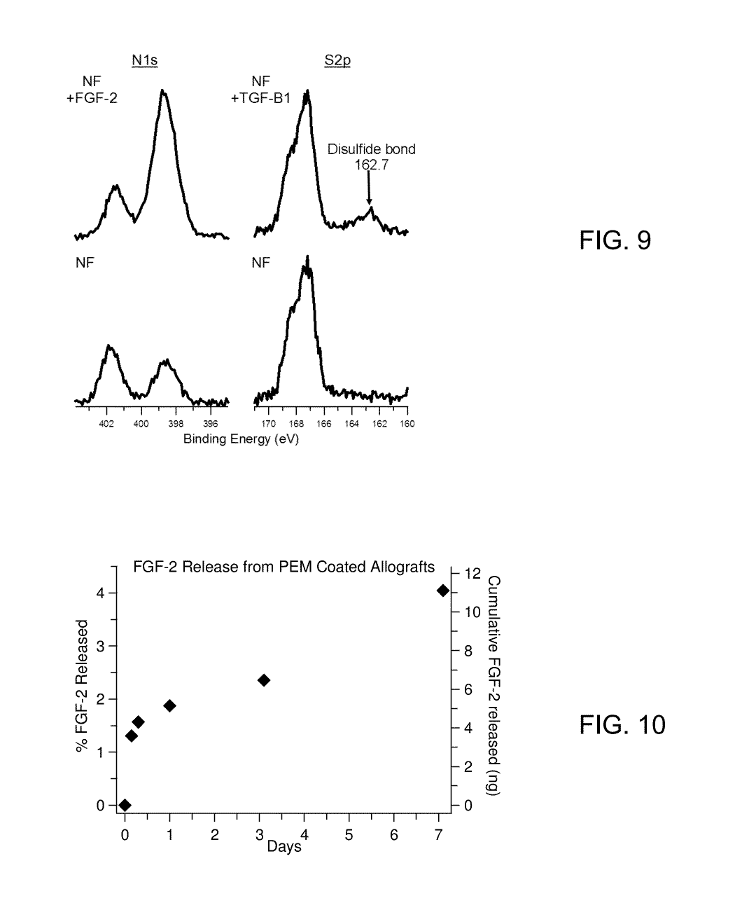

Binding of rhFGF-2 and rhTGF-β1 to PEM coated bone, PEM-modified FD and PEM-modified NF scaffolds was characterized by XPS. FIG. 9 demonstrates FGF-2 binding to PEM-modified NF scaffold through an increased contribution of amine and amide peaks in the N1s envelope. rhTGF-β1 binding was confirmed by the appearance of a disulfide bond at 162.7 eV. FIG. 10 shows kinetic release of FGF-2 on PEM coated allograft bone. Initial rhFGF-2 binding results suggested a binding efficiency ranging from 48% to 61%. This resulted in an average of 275 ng of rhFGF-2 bound to the PEM coated allografts. After a 7-day release window, only 4% of bound rhFGF-2 was released. This resulted in an optimal mitogenic dose of rhFGF-2 being present for all time points.

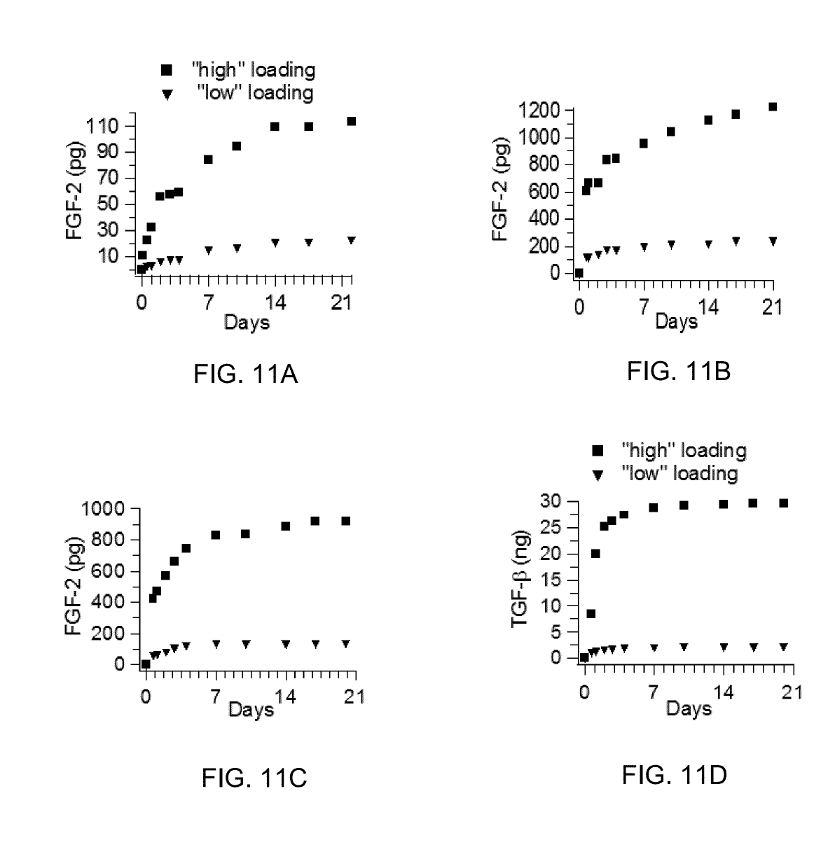

The release of bound growth factor into solution from the different coatings was measured for up to three weeks. Release kinetics of FGF-2 are shown in FIG. 11A-C. The total amount of FGF released over the three weeks varied between 0.5% and 2% of the total amount of growth factor loaded (Table 1), indicating that the coated allografts retained over 98% of the bound growth factor. Nonetheless, the growth factor released, on the order of hundreds of picograms to nanograms, result in dissolved growth factor concentrations sufficient to induce mitosis. Additionally, the FGF-2 bound to heparin-coated surfaces would also be active. The release of TGF-β from surfaces was also measured. An example of TGF-β released from nanofiber scaffolds is shown in FIG. 11D. A much larger fraction of the TGF-β was released over the three weeks than the FGF-2 fraction.

Western Blotting:

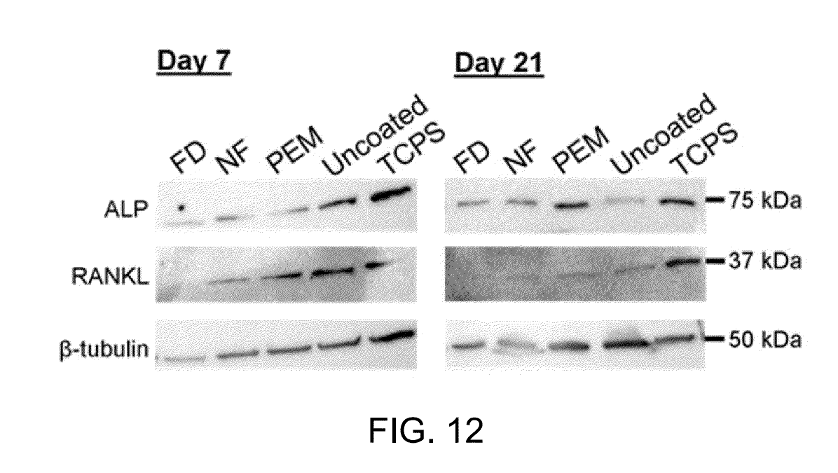

Luc-ASC osteogenic differentiation was evaluated by probing for osteogenic protein expression with Western blotting. Alkaline phosphatase (ALP) bands were observed at 75 kDa on days 7 and 21 for PEM, FD and PEM, and NF and PEM scaffolds, as seen in FIG. 12. Modified allografts displayed similarly decreased ALP expression compared to an uncoated control on day 7. By day 21, modified allografts displayed increased ALP expression compared to an uncoated control, with PEM-modified scaffolds having the highest ALP expression. Receptor activator of nuclear factor κ-B ligand (RANKL) bands were observed for NF and PEM- and PEM-modified allografts at 37 kDa on day 7 and day 21. RANKL bands of NF and PEM- and PEM-modified allografts had decreased expression by day 21 compared an uncoated control allograft. No RANKL expression was observed for FD and PEM-modified allografts at either days 7 or 21. No osteocalcin, osteopontin, or osteonectin expression was observed for any experimental group at either timepoint.

### Example 2

### In Vivo Study of Coated Bone Allografts

### Materials and Methods

Devitalized Bone Graft Transplantation:

4-mm allografts harvested from BALB/c mice are scraped to remove the periosteum, extensively washed in a 0.9% sodium chloride (NaCl) solution containing polymyxin B sulfate (500,000 units/L), neomycin (1 g), and ampicillin (3 GM) saline/antibiotic solution, double-wrapped in saline-soaked gauze and frozen at −80° C. for at least 1 week prior to coating with FD/PEM or NF/PEM. Coatings are performed under sterile conditions and allografts are returned to a temperature of −80° C. prior to being thawed for implantation. Forty-eight (n=48) BL/6 mice serve as allograft recipients. As the segmental bone grafts used in the current study are harvested from a genetically different mouse strain, allograft transplantation closely mimics the clinical situation experienced in the human population, where cortical allografts are not HLA-matched between donor and recipient. The mice are anesthetized and the right femur aseptically prepared for surgery. A 7 to 8 mm long incision is made using a lateral approach to the femur, and the midshaft femur is exposed following blunt dissection of the surrounding muscles. A 4-mm mid-diaphyseal segment is removed from the femur by osteotomizing the bone using a saw. Subsequently, a 4-mm cortical bone graft is inserted and stabilized using a 23-gauge intramedullary pin, as previously described (Xie et al. Tissue Eng. 13:435-45. 2007). The technique results in mice that are ambulatory on the operated limb within 24 hours.

The FD and PEM or NF and PEM allograft coating is evaluated with and without MSC transplantation. Allograft treatment groups are as follows: coating with MSCs (n=12); MSCs, no coating (n=12); coating, no MSCs (n=12); no coating, no MSCs (n=12). For animals receiving MSCs, culture-expanded MSCs are grown to 70% confluence and allowed to recover for 24 hours. Passages 2 and 3 are used for all experiments to assure fidelity and consistency of donor MSCs. The MSCs are trypsinized, centrifuged, and re-suspended in warmed (37° C.) lactated-Ringer's solution at 5×10⁶ cells/mL. One million cells (200 μL) are seeded onto the allografts prior to closure of the surgical wound. Animals are recovered and observed daily for 1 week. Buprenorphine is administered for analgesia. Mice are anesthetized, and radiographs of the operated femur are obtained at postoperative week three and immediately after euthanasia at postoperative week six. Euthanasia occurs at six weeks post-surgery and operated femurs are harvested. Grafted femurs are dissected to remove non-adherent musculature and formalin fixed for 48 hours, for microCT and histology.

Micro CT:

formalin-fixed femurs from all treatment groups are scanned using a μACT-80 imaging system (Scanco USA, Southeastern, Pa.) at a voxel size of approximately 10 μm. From the 2-dimensional slice images generated, an appropriate threshold is chosen for the bone voxels by visually matching thresholded areas to gray-scale images. The threshold and the volume of interest (VOI) covering the entire length of the allograft and 50 slices into the host bone at both bone graft junctions are kept constant throughout the analysis for each femur. To measure the new bone volume, contour lines are drawn in the 2-dimensional slice images to exclude the allograft and the old host cortical bone. New bone volume in a volume of interest (VOI) covering the entire length of the allograft and 1 mm of the host bone at both bone graft junctions is used as a quantitative measure of graft healing. Average cross-sectional polar moment of inertia (pMOI) at the region of the graft is evaluated based on histomorphometric and MOI programs in the Scanco system. Qualitative evidence of host-allograft union is also documented.

Tissue Processing:

Following microCT analysis, grafted femurs are decalcified in EDTA (14%) for 4-7 days. Given that the ability to detect GFP expression is highly variable in paraffin embedded tissues, 4 femurs from each treatment group are processed for frozen section and epifluorescence microscopy using a tape transfer processing method that has been previously described in detail (Jiang et al. J Histochem Cytochem. 53:59-602. 2005). Briefly, following decalcification, the femurs are soaked in 30% sucrose in PBS for 24 hours. The samples are then immersed in frozen embedding media with their posterior surfaces against the bottom of the mold to maintain consistent orientation for sectioning. The embedding media is then flash-frozen, taking care to keep the femurs flat against the base of the mold. Once frozen, the samples are stored at −80° C. until sectioning. Sectioning is performed on a cryostat and frozen sections are captured on cold adhesive tape. Sections are transferred to a cold glass microscope slide coated in a UV-light curable pressure sensitive adhesive and cured with a flash of UV light. The slides are then air dried and stored at −80° C. in the dark until analysis. Tissue sections immediately adjacent to the sections taken for GFP analysis are stained with hematoxylin and eosin (H&E) using standard techniques to allow for analysis of GFP-positive cell morphology. The 8 remaining femurs in each treatment group are processed for decalcified histologic analysis.

Histological Analysis:

Grafted femurs are embedded in paraffin blocks with the posterior sides at the bottom of the block to maintain consistent orientation and sectioned using standard techniques. Blocks are sectioned in 5 μm slices until the entire length of the femur is visible on each section. Standard H&E staining is used for analysis. Histologic analysis is performed by a single, blinded board-certified veterinary pathologist familiar with allograft models. Analysis is performed using a categorical scoring system of 0-3 (with zero being none and 3 being marked) covering the following criteria: bridging at proximal graft site, bridging at distal graft site, overall graft-host union score, graft incorporation (viable cells infiltrating graft tissue and degree of remodeling of graft), cutting cones within graft, callous formation, fibroplasia at host-graft junction, graft-associated marrow elements, graft-associated intramedullary trabecular bone formation, and inflammation grade. Type of inflammation, if present, is also described (i.e., granulomatous, neutrophilic, lymphocytic plasmacytic, and the like).

Statistical Analysis:

Data are reported as mean±standard error of the mean. Statistical significance between micro-CT-derived pMOI and new bone formation, histologic scores of allograft incorporation, and radiographic evidence of healing is determined with a one-way ANOVA and a Tukey's post hoc test. Mann-Whitney non-parametric tests are used to make the statistical comparisons of non-normal data. Structure-function relationships are determined by calculating Pearson correlation coefficients between mechanical properties and new bone volume around grafts or average cross-sectional pMOI. All statistical comparisons are made with SigmaStat (Systat Software, Inc., San Jose, Calif.) at using α=0.05.

Results

Animals receiving FD and PEM- or NF and PEM-coated allografts with growth factors and seeded with stem cells are expected to have the best overall allograft incorporation compared to control and PEM only-coated allografts. Improvement in graft healing is manifested as a significant increase in new bone volume adjacent to the grafted femurs, concomitant with increases in pMOI of the grafted femurs and most favorable overall histologic indices of graft incorporation. Both groups receiving MSCs will have significantly lower inflammation scores as seen on histology when compared to groups that do not receive MSCs.

The foregoing Examples demonstrate coated bone surfaces that improve healing of bone allografts. The Examples provide three distinct tissue engineered coatings on cortical bone in order to mimic the biological function of the periosteum. First, chitosan nanofibers were directly electrospun on murine bone allografts and subsequently modified with TMC and heparin polyelectrolyte multilayers using a layer-by-layer deposition technique. The disclosure provides the first demonstration of the direct modification of bone with a polymer nanofiber to form a biomimetic synthetic periosteum. Second, TMC and heparin polyelectrolyte multilayers were deposited on murine cortical bone coated with freeze-dried chitosan scaffold. Third, TMC and heparin polyelectrolyte multilayers were directly deposited onto murine cortical bone.

The FD and PEM- and NF and PEM-coated bone surfaces did not undergo morphological changes through the several aqueous processing steps. The coated bone surfaces supported ASC proliferation for at least 21 days when cultured in vitro. The coated bones of the disclosure can locally deliver growth factors and stem cells in order to improve host-allograft union. The disclosure thus provides coated bone surfaces possessing the ability to recover lost osteogenic, osteoconductive, and osteoinductive characteristics, e.g., for devitalized bone allografts, through creation of a biomimetic periosteum. The methods can be utilized in other tissue engineering and regenerative medicine applications.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 nm” is intended to mean both 10 nm and “about 10 nm.”

All documents cited in the Detailed Description are, in relevant part, incorporated herein by reference; the citation of any document is not to be construed as an admission that it is prior art with respect to the disclosure. To the extent that any meaning or definition of a term in this document conflicts with any meaning or definition of the same term in a document incorporated by reference, the meaning or definition assigned to that term in this document shall govern.

While particular embodiments of the disclosure have been illustrated and described, it will be obvious to those skilled in the art that various other changes and modifications can be made without departing from the spirit and scope of the invention. It is therefore intended to cover in the appended claims all such changes and modifications that are within the scope of this invention.

What is claimed:

1. A coated bone comprising a coating comprising (a) a porous polysaccharide scaffold or a plurality of polysaccharide nanofibers; and (b) a polyelectrolyte multilayer composition.

2. The coating of claim 1, comprising a porous polysaccharide scaffold freeze-dried onto the bone surface or a plurality of polysaccharide nanofibers electrospun onto the bone surface.

3. The coating of claim 1, wherein the polysaccharide is selected from the group consisting of chitosan, alkylated chitosan, acylated chitosan, and combinations thereof.

4. The coating of claim 1, wherein the polyelectrolyte multilayer composition is contacting the porous polysaccharide scaffold surface or plurality of polysaccharide nanofibers.

5. The coating of claim 4, wherein the porous polysaccharide scaffold or the plurality of polysaccharide nanofibers is neutralized with a strong base and water-insoluble before addition of the polyelectrolyte multilayer composition.

6. The coating of claim 1, wherein the polyelectrolyte multilayer composition comprises a polyanion selected from the group consisting of a glycosaminoglycan, an anionic polysaccharide, a synthetic anionic polymer, and combinations thereof.

7. The coating of claim 6, wherein the polyelectrolyte multilayer composition comprises a glycosaminoglycan selected from the group consisting of heparin, heparan sulfate, hyaluronan, chondroitin sulfate, dermatan sulfate, keratan sulfate, and combinations thereof.

8. The coating of claim 6, wherein the polyelectrolyte multilayer composition comprises an anionic polysaccharide or synthetic anionic polymer selected from the group consisting of sulfated chitosan, dextran sulfate, a fucan, a carrageenan, a pectin, poly(styrene sulfonate), and combinations thereof.

9. The coating of claim 1, wherein the polyelectrolyte multilayer composition comprises a polycation selected from the group consisting of chitosan, alkylated chitosan, acylated chitosan, poly-lysine, polyethylenimine, and combinations thereof.

10. The coating of claim 1, wherein the polyelectrolyte multilayer composition comprises N,N,N,-trimethyl chitosan and heparin.

11. The coating of claim 1, further comprising a therapeutic agent selected from the group consisting of a growth factor, a progenitor cell, a hormone, an anti-inflammatory agent, an antibiotic, a polynucleotide, a chemotherapeutic agent, and combinations thereof.

12. The coating of claim 11, comprising a growth factor bound to a polyanion in the polyelectrolyte multilayer composition.

13. A method of stabilizing and delivering a therapeutic agent comprising administering the coated bone of claim 11 to a subject in need thereof.

14. The coated bone of claim 1, wherein the bone is a bone graft.

15. The bone graft of claim 14, wherein the bone graft is an allograft.

16. A method of transplanting bone comprising administering the bone graft of claim 14 to a subject in need thereof.

17. A method of coating a bone surface comprising contacting the bone with (a) a porous scaffold-forming polysaccharide or a plurality of polysaccharide nanofibers and (b) a polyelectrolyte multilayer composition.

18. The method of claim 17, wherein the porous scaffold-forming polysaccharide or plurality of polysaccharide nanofibers is deposited onto the bone surface, and the polyelectrolyte multilayer composition is applied onto the porous scaffold-forming polysaccharide or plurality of polysaccharide nanofibers.

19. The method of claim 18, wherein the periosteum of the bone surface is removed before depositing the porous scaffold-forming polysaccharide or plurality of polysaccharide nanofibers onto the bone surface.

20. The method of claim 17, wherein the porous scaffold-forming polysaccharide is deposited using freeze-drying to form a porous polysaccharide scaffold or the plurality of polysaccharide nanofibers is deposited using electrospinning.

21. The method of claim 17, wherein the porous scaffold-forming polysaccharide or the plurality of polysaccharide nanofibers is treated with a strong base to neutralize the polysaccharide and make it water-insoluble before depositing the polyelectrolyte multilayer composition.

22. The method of claim 17, wherein the polysaccharide is selected from the group consisting of chitosan, alkylated chitosan, acylated chitosan, and combinations thereof.

23. The method of claim 17, wherein the polyelectrolyte multilayer composition comprises a polyanion selected from the group consisting of a glycosaminoglycan, an anionic polysaccharide, a synthetic anionic polymer, and combinations thereof.

24. The method of claim 17, wherein the polyelectrolyte multilayer composition comprises a polycation selected from the group consisting of chitosan, alkylated chitosan, acylated chitosan, poly-lysine, polyethylenimine, and combinations thereof.

25. The method of claim 17, wherein the polyelectrolyte multilayer composition comprises N,N,N,-trimethyl chitosan and heparin.

26. A kit comprising (a) a porous scaffold-forming polysaccharide or nanofiber-forming polysaccharide; (b) a polyelectrolyte multilayer-forming composition; and (c) instructions for coating a bone surface with (a) and (b).
